(19) United States
(12) Patent Application Publication
(10) Pub. No.: US 2026/0009013 A1
(43) Pub. Date: Jan. 8, 2026

(54) PROCESSING AND FABRICATION OF COMPOSITE SILK-BASED PLASTICS

(71) Applicant: Trustees of Tufts College, Medford, MA (US)

(72) Inventors: David L. Kaplan, Concord, MA (US); Junqi Wu, Medford, MA (US); Rodrick Wiggins, Northfield, OH (US); Gerard Kugel, Lincoln, NE (US)

(21) Appl. No.: 19/239,426

(22) Filed: Jun. 16, 2025

Related U.S. Application Data
(63) Continuation of application No. PCT/US23/84801, filed on Dec. 19, 2023.
(60) Provisional application No. 63/476,167, filed on Dec. 19, 2022.

(51) Int. Cl. C12N 11/04; C07K 14/435; C12N 9/64

(52) U.S. Cl. CPC C12N 11/04; C07K 14/43586; C12N 9/6464; C12Y 304/21069

(57) ABSTRACT

Silk cocoons are used to directly prepare biodegradable silk articles by a thermoplastic method. Combining the silk cocoons with plasticizers, inorganic salts, and biopolymers can add bulk, reduce costs, maintain degradability and provide tunability of the material properties of the silk articles. The on-demand degradation of these silk articles is achieved by exposure to moisture, enzyme solutions or activation of embedded proteases within the article.

### CLAIM TO PRIORITY

This application claims benefit of and is a continuation of International Patent Application No. PCT/US2023/084801 (Attorney Docket. No. 2095.0583), filed Dec. 19, 2023, and entitled “PROCESSING AND FABRICATION OF COMPOSITE SILK-BASED PLASTICS,” International Pub. No. 2024137617, which is hereby incorporated by reference in its entirety for all purposes.

International Patent Application No. PCT/US2023/084801 claims the benefit of priority to the following provisional application, which is hereby incorporated by reference in its entirety for all purposes: U.S. Provisional Application 63/476,167 (Attorney Docket No. 2095.0423), filed Dec. 19, 2022.

### STATEMENT REGARDING FEDERALLY SPONSORED RESEARCH

This invention was made with government support under FA9550-20-1-0363 awarded by US Air Force/Air Force Office of Scientific Research. The government has certain rights in the invention.

### FIELD OF THE INVENTION

The disclosed technology is generally directed to molding of articles. More particularly the technology is directed to hot pressing of silk-based plastic articles.

### BACKGROUND OF THE INVENTION

Current commodity plastics such as toothbrush handles made from synthetic polymers like polypropylene and polyethylene accumulate in the environment at an annual rate of 1 billion per year without significant degradation, resulting in severe and growing environmental burdens. Most commodity plastics used in our daily life like toothbrush handles are currently manufactured from polyethylene (PE) and polypropylene (PP). These polymers are among the most used plastics in consumer goods because they are thermoplastic, moldable, and cost-effective, with mature manufacturing infrastructure. For oral applications, they also have excellent physical, mechanical, and thermal properties. They are resistant to acids, bases, humidity, and are nonconductive.

However, PE and PP are made from petroleum-based raw materials and have a tremendous carbon footprint when produced and used in commodity products. The widespread manufacturing of PP is predicted to contribute over 1 billion tons of CO₂ over the next 30 years. These materials are also non-biodegradable, contributing huge environmental burdens. Approximately one billion toothbrushes are thrown away annually, ending up in the landfills, soil, ocean, and ecosystems. These petroleum products accumulate over time and if not disposed of properly absorb and release toxic chemicals (metals, pesticides, etc.) in the process.

Recycling can be used to reduce the environmental impact of synthetic plastics, but this requires special recycling programs, separation of the different parts like the bristles and handles and is generally inefficient.

Using biodegradable, natural materials to replace synthetic plastics has attracted attention as a sustainable approach to new materials. Current commercially available alternatives include bamboo and wood. Bamboo has served as a sustainable material for centuries because it is durable and plentiful. In an appropriate context, bamboo is flexible and inexpensive. Using toothbrush handles as an example, there are inherent flaws with bamboo toothbrush handles that pose barriers to a sustainable impact. Depending on the individual manufacturer, the quality of the toothbrush handle can differ due to natural porous components within wood and bamboo. This loss of integrity over time can lead to blistering of the wood or bamboo, which leads to irritation of the brush head against the oral mucosa, as well as resulting in bristles falling out of the brush head. In addition to this problem, during manufacturing and machining of the toothbrush handles, the bamboo loses its natural antibacterial properties in the bark, leading to bacteria buildup on the handle and brush head. In addition, bamboo or wooden based toothbrush handles are composed mainly of cellulose which is prone to absorbing water, leading to undesirable structural changes of the handles.

### BRIEF SUMMARY OF THE INVENTION

In an aspect, the present disclosure provides an article comprising a thermoplastically-molded silk cocoon from the species Bombyx mori. The thermoplastically-molded silk cocoon has undergone plastic deformation.

In another aspect, the present disclosure provides a method of making an article. The method includes the following steps: (a) shredding cocoons into silk strips; (b) packing the silk strips into a mold; (c) optionally adding between 1 wt. % and 50 wt. % of an additive to the silk strips in the mold; and (d) pressing the silk strips in the mold, thereby forming the article.

In a further aspect, the present disclosure provides a method of making an article. The method includes the following steps: (a) shredding cocoons into silk strips; (b) optionally coating the silk strips with between 1 wt. % and 50 wt. % of an additive; (c) packing the silk strips in the mold; and (d) pressing the silk strips in the mold at an elevated temperature, thereby forming the article.

In yet another aspect, the present disclosure provides a method of processing an article comprising a thermoplastically-molded silk cocoon. The method includes contacting the article with a protease that selectively dissolved the thermoplastically-molded silk cocoon.

In yet a further aspect, the present disclosure provides a method of processing an article comprising a thermoplastically-molded silk cocoon. The method includes embedding and maintaining the article in a moist plant soil.

In still yet a further aspect, the present disclosure provides a method of processing an article comprising a thermoplastically-molded silk cocoon including an embedded protease. The method includes contacting the article with an aqueous solvent to activate the embedded protease and dissolve the thermoplastically-molded silk cocoon.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. The process of making toothbrush handles from silk cocoons. (A) Illustration of the thermoplastic molding process. Silk cocoons were first shredded into small pieces and packed in a mold followed by thermoplastic molding with glycerol added as plasticizer. (B-D) Digital images of toothbrush handles prepared from thermoplastic molded silk cocoons 163 mm×12 mm×7 mm (right side of each image) compared with wooden toothbrush handles 192 mm×13 mm×8 mm (left side).

FIG. 2 Composites made from one-step solid mixing of silk cocoons with (A) 20% cellulose, (B) 50% cellulose, (C), 10% hydroxyapatite (HAP), (D), 20% HAP, and (E) 10% chitosan.

FIG. 3. Biodegradation of thermoplastic molded silk bars at two protease concentrations 1 mg/mL and 5 mg/mL at 37° C.

FIG. 4. Degradation test (A) Weight loss of the thermoplastic molded toothbrush handles in 1 mg/mL and 5 mg/mL protease XIV DPBS solutions at 37° C. The weight was measured by taking the samples out of the degradation solution and the surface water was removed using a paper towel before weighing the sample. (B) Weight measurement of the thermoplastic molded toothbrush handles in 5 mg/mL protease XIV DPBS solutions at 37° C. The samples were collected from the degradation solution, rinsed with water several times and dried overnight in the fume hood before the weight was measured.

FIG. 5. Degradation of thermoplastic molded silk bars with protease XIV embedded in the handles in DPBS at 37° C.

FIG. 6. Degradation test. (A) Weight loss of thermoplastic molded silk bars with protease embedded at 1 wt % and 5 wt % when immersed in DPBS at 37° C. The weight was measured by taking the samples out of the degradation solution and the surface water was removed using a paper towel before weighing the sample. (B) Weight measurement of the silk bars with 5% protease XIV embedded degrading at 37° C. The samples were collected from the degradation solution, rinsed with water several times and dried overnight in the fume hood before the weight was measured.

FIG. 7. Degradation of thermoplastic molded silk bars in plant soil with 160 mL of room temperature water added each week.

### DETAILED DESCRIPTION OF THE INVENTION

Silk can be a useful alternative material to replace commodity plastics because silk is biodegradable and has excellent mechanical properties. Using silk to manufacture hard or dense materials can be achieved by the following methods: 1) direct assembly from regenerated silk aqueous solution, 2) Hexafluoro-2-propanol (HFIP) solvent method, and 3) regenerated silk amorphous powder thermoplastic molding. For commodity plastic replacement, mass production is required, thus, these three methods have limitations. Regenerated silk production may be costly and inefficient for commodity products requiring multiple processing steps including degumming dissolution, purification, and freeze drying. HFIP is a toxic solvent, thus processing requires extra costs to deal with the chemical. In addition, regenerated silk aqueous solutions require low temperature storage and transportation, which is particularly challenging when dealing with large quantities for commodity products. Direct thermoplastic molding of silk amorphous powders avoids dealing with the storage of large quantities of silk aqueous solution, yet, generating the regenerated powder is still time-consuming and costly due to the extraction and reprocessing steps still involved.

Herein is disclosed a new process to generate silk-based plastic articles for consumer goods and related needs using natural, biopolymer-based components through thermoplastic molding. In addition, the plastic articles can be processed to offer on-demand degradation. The articles are prepared directly from silk cocoons that are thermoplastically (high temperature and pressure) transformed into biodegradable plastic articles, for example, in the specific form of toothbrush handles. The on-demand degradation of these plastic articles is demonstrated in different ways, including activation of sequestered proteases that are embedded during the processing and activated on-demand to initiate the degradation. The approaches described here demonstrate a new approach to plastic article formation and to their degradation that offers widespread options for future consumer materials, maintaining utility and function of the articles as parts and devices, while reducing environmental impact. These parts can be generated directly from the silk cocoons avoiding downstream purification processes as well as by combining the cocoons with other sustainable materials (e.g., calcium salts, cellulose) to add bulk, reduce costs, maintain degradability and provide a diverse set of material properties (e.g., stiffer and stronger materials, softer and more pliable materials, etc.).

Disclosed herein is a new method to make silk-based commodity plastics or silk-based plastic articles like toothbrush handles through direct thermoplastic molding of silk cocoons. In this method, Bombyx mori cocoons or other type of silk cocoons are shredded into small pieces, for example using a paper shredding machine. The finer the pieces are, the more efficient the thermoplastic molding that can be achieved. The shredded cocoons are packed into predesigned molds and then followed by hot pressing at elevated pressure (e.g., 632 MPa) up to 1000 MPa and elevated temperature (e.g., between 25° C. and 200° C.) for at least 1 s to form the silk-based plastic article. Below approximately 50 MPa, the cocoons exhibit elastic deformation. The thermoplastic pressures, temperatures, and molding times can be expanded to cover a range of conditions. For example, the cocoon shreds can be compressed between 100 MPa and 1000 MPa, between 200 MPa and 900 MPa, between 300 MPa and 800 MPa, between 400 MPa and 700 MPa, between 500 MPa and 700 MPa, or between 600 MPa and 700 MPa.

Glycerol can be mixed with the cocoons as a plasticizer in the range of 1 wt % to 50 wt % to promote the particle fusion process in making the silk-based plastic articles. Other plasticizers like water, low molecular weight silk, or sericin can also be used to promote the fusion process.

After hot pressing, the silk-based plastic articles are cooled to room temperature and then machined to make the desired shape, such as toothbrush handles. Alternatively, the mold can be used to directly match the final part, thus, avoiding post-processing machining, loss of material and added time/cost. Note, any waste material from the process can be added into the materials used in the next run, thus, achieving near zero waste in the overall process to maximize efficiency, reduce costs and avoid waste production.

The material properties of the silk-based articles can be characterized as follows. In some embodiments, the density of the articles can be greater than 1 g/cm³. Higher density articles have an aesthetic heft pleasing to consumers and are robust towards usage. In other embodiments, the density of the silk-based plastic articles can be less than 1 g/cm³. The articles can be made with hollow cores, embedded gas bubbles or microcavities to reduce the density of the articles. Articles with low density can float on water, thereby facilitating separation for disposal or recycling.

In certain embodiments, the hardness of the article can be between 30 and 100 on the Shore D scale, between 40 and 90 on the Shore D scale, 50 and 80 on the Shore D scale, between 60 and 70 on the Shore D scale, or any combination of the lower and upper limits of these ranges not expressly recited.

It is known that silk materials are robust at low temperatures and do not fail even at −80° C. In some embodiments, the article can have a brittle temperature, or a temperature at which 50% of articles fail, between 0° C. and −20° C., between −20° C. and −40° C., or between −40° C. and −80° C.

The ability of the article to bend, or flexural modulus, is a property that can be tuned based on the intended application of the article. In the instance of a toothbrush handle, the flexural modulus should be selected for limited bending. In some embodiments, the article can have a flexural modulus between about 1 and 40 GPa. In other embodiments, the silk-based plastic article can have a flexural modulus between about 1 and 5 GPa, between 1 and 10 GPa, between 5 and 30 GPa, between 10 and 20 GPa, or between 35 and 40 GPa.

The silk-based plastic article can include fillers or hydrophobic coatings to control the water absorbency. In one example, silk can be fluorinated as disclosed in WO2022147483A2 and WO/2021/195,445, incorporated herein in their entirety, and the fluorinated silk can be incorporated into the silk-based plastic article. In some embodiments, the silk-based plastic article can have a water absorbency of between 1 wt. % and 30 wt. %, according to the ASTM D570 test. In other embodiments, the silk-based plastic article can have a water absorbency, of between 5 wt. % and 25 wt. %, or between 10 wt. % and 20 wt. %, according to the ASTM D570 test.

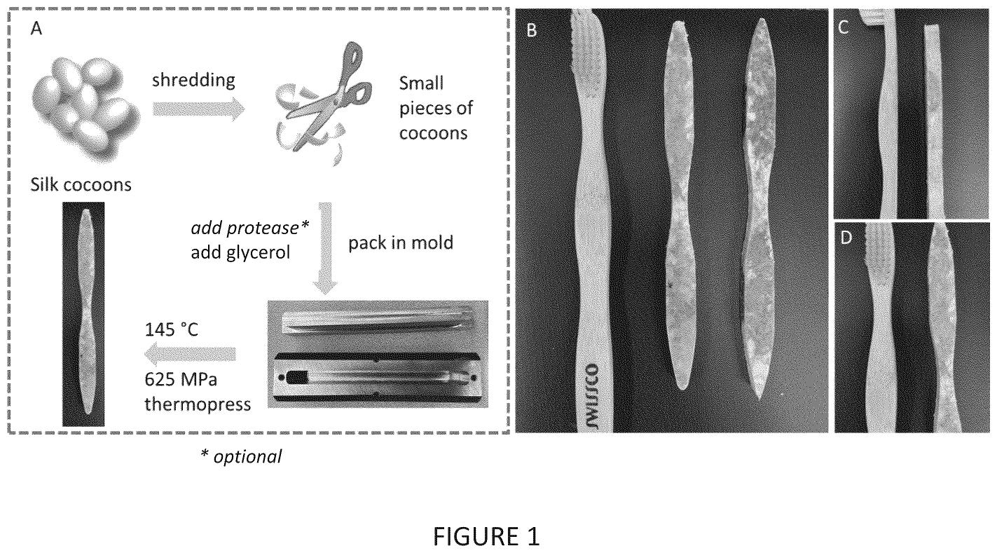

FIG. 1A illustrates the process to generate silk-based plastic articles by thermoplastic molding. In this example, biodegradable silk cocoon toothbrush handles are formed by the process as disclosed. The silk cocoon pieces are fed into a shredding machine to generate small pieces of cocoons. The smaller the size of the cocoon pieces, the easier to fill the mold for thermoplastic molding. Optionally, protease can be added to the silk shreds before molding. To enable better thermoplastic molding, plasticizers like glycerol may be added (for example, 1-50 wt %).

FIGS. 1B and 1C shows silk toothbrush handles or bars prepared through thermoplastic molding of silk cocoons with 5 wt % glycerol. The molded bars were machinable to desired shape. These new silk handles were compared with commercially available bamboo toothbrush handles. The silk handles were more hydrophobic, with similar density, and are nicely textured as shown in the picture.

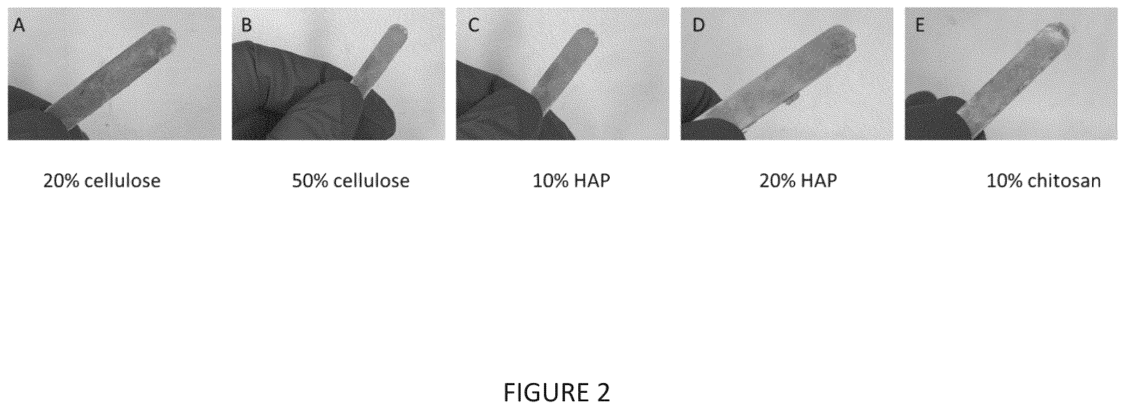

FIG. 2 shows that articles can be fabricated from silk-based composite plastics by one-step solid mixing of biopolymer powders with silk cocoon strips upon thermoplastic molding. To further reduce cost and enhance mechanical strength, the silk cocoons can be mixed with cellulose powders of up to 100 wt % (1:1 ratio), chitosan or chitin powders of up to 100 wt %, and hydroxyapatite (HAP) nanoparticles (among many other options) of up to 100 wt %, followed by thermoplastic molding to obtain plastic composites that can be used as replacements of commodity plastics. The article made this way maintains mechanical integrity and can be used to replace the current commodity plastics with good degradation ability and reduced cost.

The biodegradability of these silk articles as an example was demonstrated using three methods.

### Method 1

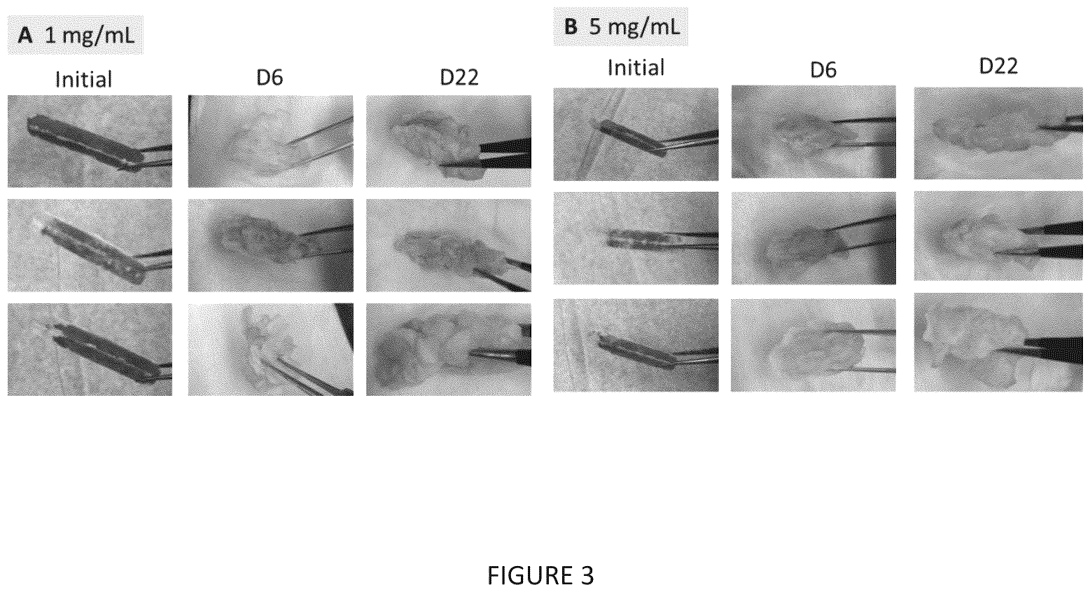

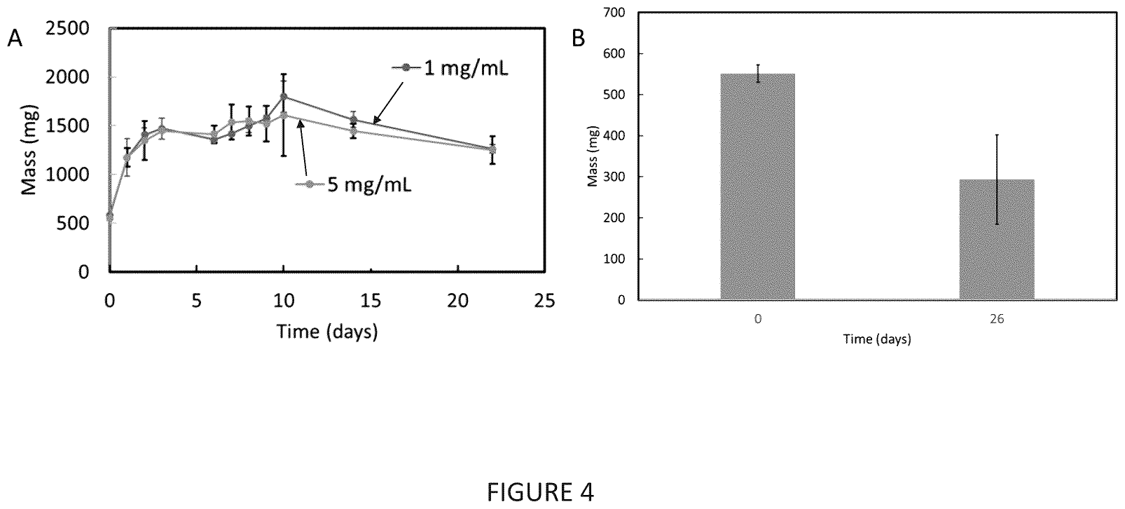

In the first method (FIGS. 3-4), the toothbrush handles can be immersed in a protease solution with a concentration of more than 0 mg/mL, for example 1 mg/mL or 5 mg/mL. The degradation rate correlates to the dosing of enzymes and the type of enzyme. The protease can include protease XIV, a-chymotrypsin, proteinase K, and papain. Other proteases can also be considered depending on commercial availability, including those from extremophiles.

In one example, the samples were immersed in protease XIV aqueous solutions at two concentrations, 1 mg/mL and 5 mg/mL. The morphology and weight changes were monitored over a month, and it was found that the handles were broken into soft fibers within a week and continued to demonstrate weight decrease over a month. FIG. 4A shows the weight changes of the silk handles, where the weight initially increased over the first 10 days due to water absorption and swelling, followed by a significant weight decrease. FIG. 4B shows the dry weight changes of the silk handles after 26 days of degradation study. Thus, the toothbrush handles can be degraded by immersion into protease solutions. In commercial use, a protease powder can optionally be supplied with the product to customers to conduct degradation at the end of the product use cycle.

### Method 2

In a second method, the cocoons are shredded and then the protease is added. In one embodiment, protease powders can be mixed with the shredded silk cocoons pre-processing. Next, the plasticizer is added to the combination of shredded silk cocoons and the proteases. The solid mix is then thermoplastically molded to ensure the protease is embedded inside of the toothbrush, but the enzyme remains inactive due to the low water content.

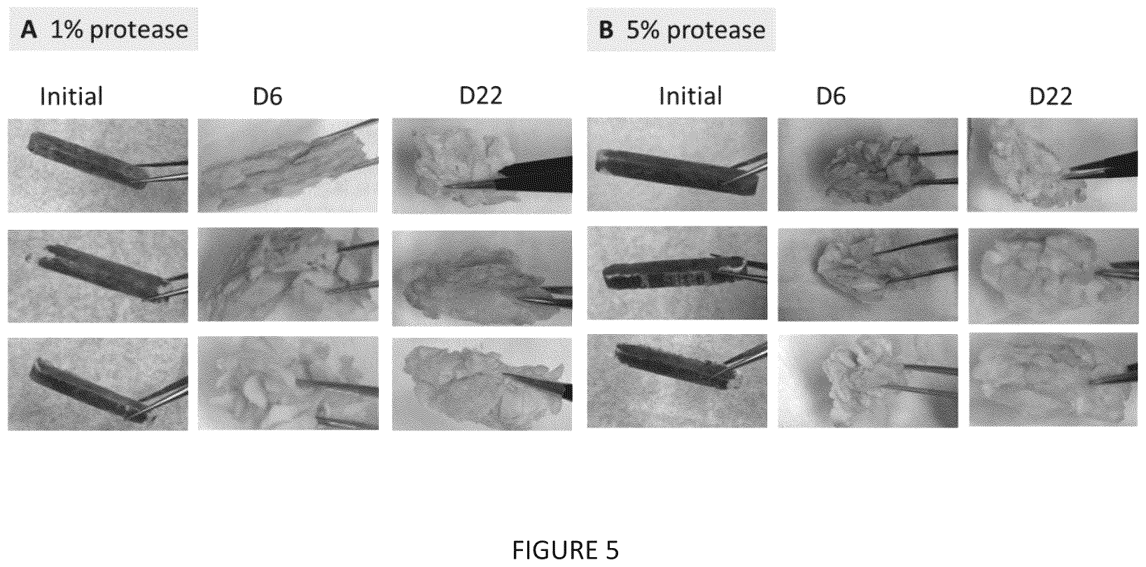

The silk-based plastic articles made this way can be immersed in water for rapid biodegradation. For maintained their integrity, texture and hydrophobicity as well as mechanical strength with the protease addition. As can be seen in FIG. 5, these protease-embedded silk bars showed significant degradation within a month (22 days), thus, the protease activities were maintained after thermoplastic molding.

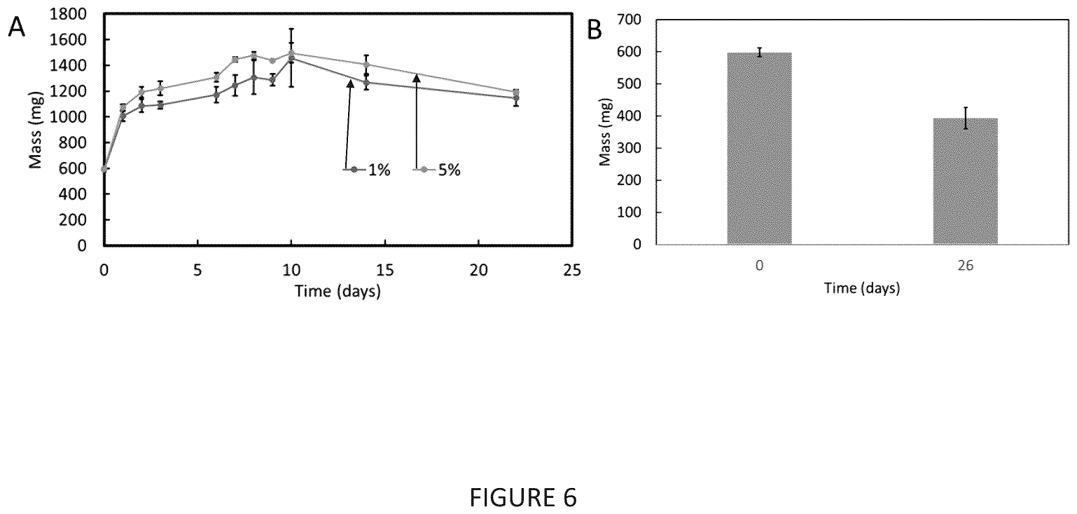

FIG. 6 shows the weight changes of articles formed as silk bars over time. The weight change shows similar trends as degradation in protease solution. The silk bars absorb water and the weight reached the maximum at day 10 and then started decreasing mass. Using this method, no additional protease powder is needed for product degradation. The dry weight change was monitored for 26 days, showing that dry biomass of the silk handles decreased upon degradation.

### Method 3

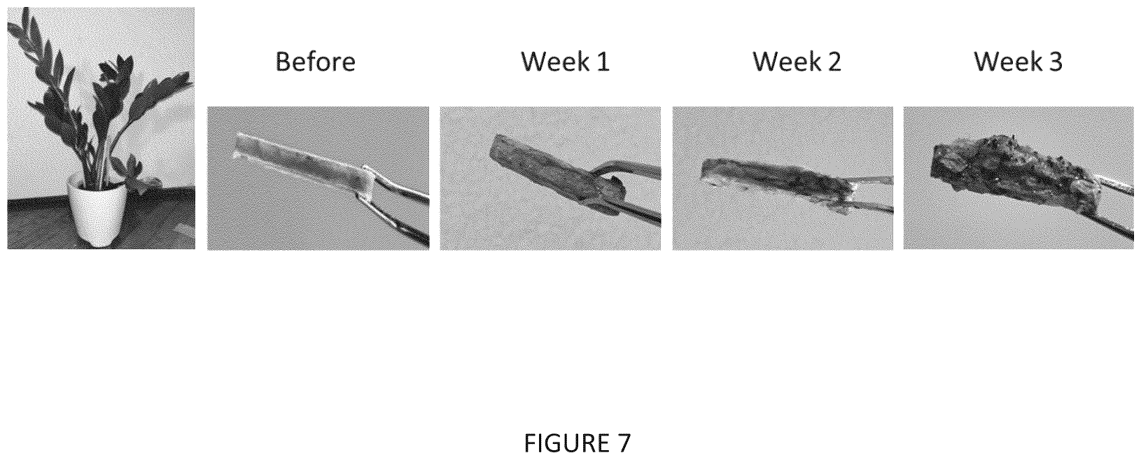

In the third method, thermoplastic molded silk bars were immersed into plant soil to induce biodegradation (FIG. 7). The silk bars showed morphological changes within 3 weeks. This experiment shows biodegradation at home by immersion in plant soils.

Disclosed herein are articles including a thermoplastically-molded silk cocoon from the species Bombyx mori as well as methods of making and processing the articles. Surprisingly, it was found that compressing silk cocoons that were processed into portions (e.g., strips) under conditions of high temperature and/or high pressure resulted in their thermoplastic deformation into a silk-based plastic. Silk-based plastics formed by this method are suitable for thermomolding under conditions of elevated temperature and/or pressure, and furthermore, the properties of these silk-based plastics can be altered or augmented by the inclusion of additives during the process of making it, such as to alter the flexibility or durability of the silk-based plastic or articles made from it. Other impacts that additives may have include impacts on speed of degradation, surface feel, visual appearance, or aroma, and certain additives may also have an effect on the ultimate cost of the article, or methods of making or processing it.

A truly significant advancement in this field is the finding that the silk-based plastics can degrade on-demand. For example, the silk-based plastic can be degraded under certain conditions, such as exposure to a microbial and enzyme rich soil environment, or exposure to a protease solution. Embedding proteases into the silk-based plastic enables rapid, on-demand degradation upon exposure to an aqueous environment, such as immersion into water.

Throughout this disclosure, a toothbrush handle is used to exemplify an article that is possible to obtain using the methods disclosed herein, however, it is understood that any article may be made with the silk-based plastics made by any of the methods disclosed herein. Articles made from silk-based plastics may be further finished to specification to meet user requirements, such as with robotic or CNC trimming, adding attachments, bonding with other parts, assembling with other parts, coating the article, adding branding to the article, shrink wrapping the article, and the like.

In embodiments of the article, the thermoplastically-molded silk cocoon has undergone plastic deformation. In some embodiments, the thermoplastically-molded silk cocoon may further include a plasticizer, such as glycerol, water, low molecular weight silk fibroin, or sericin, or combinations thereof, and the plasticizer may be in an amount by weight of between 1% and 50%. In some embodiments, the article further includes an additional biopolymer that is not present within the thermoplastically-molded silk cocoon. In some embodiments, the article further includes at least one of a calcium salt, a cellulose, chitosan, chitin, hydroxyapatite (HAP) nanoparticles, a colorant, or an aromant. In some embodiments, the article further includes an embedded protease, such as protease XIV, a-chymotrypsin, proteinase K, and papain. The density of the article may be between 0.6 g/cm³ and 1.4 g/cm³, including but not limited to at least 0.6 g/cm³, at least 1.0 g/cm³, at least 1.3 g/cm³, or at least 1.5 g/cm³, or at most 1.6 g/cm³, at most 1.0 g/cm³, or at most 0.6 g/cm³.

In some embodiments, the article may include a density reducing additive having a lower bulk density than the thermoplastically-molded silk cocoon, thereby providing the article with a density that is lower than the bulk density of the thermoplastically-molded silk cocoon. For example, the density reducing additive may be entrapped gas. In other embodiments, density reduction is achieved via a hollow core or a microcavity. In embodiments, the hardness of the article may be between 30 and 100 on the Shore D scale, between 40 and 90 on the Shore D scale, between 50 and 80 on the Shore D scale, or between 60 and 70 on the Shore D scale, including but not limited to at least 30 on the Shore D scale, at least 60 on the Shore D scale, or at least 90 on the Shore D scale, or at most 100 on the Shore D scale, at most 70 on the Shore D scale, or at most 40 on the Shore D scale. In some embodiments, the flexural modulus of the article is between 1 and 40 GPa, between about 1 and 5 GPa, between 1 and 10 GPa, between 5 and 30 GPa, between 10 and 20 GPa, or between 35 and 40 GPa, including but not limited to at least 1 GPa, at least 15 GPa, at least 20 GPa, or at least 30 GPa, or at most 40 GPa, at most 20 GPa, at most 5 GPa, or at most 1 GPa. In some embodiments, the water absorbency of the article is between 1 wt. % and 30 wt. % according to the ASTM D570 test, including but not limited to, at least 1 wt. %, at least 15 wt. %, at least 20 wt. %, or at least 30 wt. %, or at most 30 wt. %, at most 25 wt. %, at most 10 wt. %, at most 5 wt. %, or at most 1 wt. %. In some embodiments, the article has a brittle temperature between 0° C. and −20° C., between −20° C. and −40° C., or between −40° C. and −80° C., including but not limited to at least −80° C., at least −60° C., at least −10° C., or at least 0° C., or at most 0° C., at most −30° C., at most −60° C., or at most −80° C.

In embodiments, a method of making an article may include shredding cocoons into silk strips, packing the silk strips into a mold, optionally adding between 1 wt. % and 50 wt. % of an additive to the silk strips in the mold, and pressing the silk strips in the mold at an elevated temperature, thereby forming the article. The additive may be a plasticizer, cellulose, chitosan, chitin, hydroxyapatite, an enzyme, a colorant, an aromant, or a combination thereof, wherein the enzyme is selected from the group comprising protease XIV, a-chymotrypsin, proteinase K, and papain. The plasticizer may be added in the amount of between 1 wt. % and 50 wt. %, including but not limited to, at least 1 wt. %, at least 15 wt. %, at least 20 wt. %, or at least 50 wt. %, or at most 50 wt. %, at most 25 wt. %, at most 10 wt. %, at most 5 wt. %, or at most 1 wt. %. The plasticizer may be selected from the group consisting of glycerol, water, low molecular weight silk fibroin, and sericin. Pressing may be performed at an elevated temperature between 25° C. and 200° C., including but not limited to at least 25° C., at least 50° C., at least 140° C., or at least 200° C., or at most 200° C., at most 170° C., at most 90° C., or at most 25° C. Pressing may be performed at 632 MPa and 145° C. for 15 minutes. The method may further include optionally adding a filler to the silk strips.

In embodiments, a method of making an article may include shredding silk cocoons into silk strips, optionally coating the silk strips with between 1 wt. % and 50 wt. % of an additive, packing the silk strips into a mold, and pressing the silk strips in the mold at an elevated temperature, thereby forming the article. In embodiments, the additive is a plasticizer, cellulose, chitosan, chitin, a calcium salt, hydroxyapatite, an enzyme, a colorant, an aromant, nothing, or a combination thereof. The plasticizer may be added in the amount of between 1 wt. % and 50 wt. %, including but not limited to, at least 1 wt. %, at least 15 wt. %, at least 20 wt. %, or at least 50 wt. %, or at most 50 wt. %, at most 25 wt. %, at most 10 wt. %, at most 5 wt. %, or at most 1 wt. %. The plasticizer may be selected from the group consisting of glycerol, water, low molecular weight silk fibroin, and sericin. The elevated temperature may be between 100° C. and 200° C., including but not limited to at least 100° C., at least 140° C., or at least 200° C., or at most 200° C., at most 170° C., or at most 100° C. Pressing may be between 100 MPa and 1000 MPa, including but not limited to at least 100 MPa, at least 500 MPa, or at least 1000 MPa, or at most 1000 MPa, at most 800 MPa, at most 400 MPa, or at most 100 MPa. In embodiments, the coating may be hydrophobic. In some embodiments, the method may further include optionally adding a filler to the silk strips.

In embodiments, a method of processing an article including a thermoplastically-molded silk cocoon may include contacting the article with a protease that selectively dissolves the thermoplastically-molded silk cocoon. In other embodiments, a method of processing an article including a thermoplastically-molded silk cocoon may include embedding and maintaining the article in a moist plant soil. In either of the immediately preceding methods, the article may be any of the articles disclosed herein.

In embodiments, a method of processing an article including a thermoplastically-molded silk cocoon comprising an embedded protease may include contacting the article with an aqueous solvent to activate the embedded protease and dissolve the thermoplastically-molded silk cocoon. The article may be any of the articles disclosed herein having an embedded protease.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Examples

### Experimental Methods

Fabrication of Silk Cocoon-Based Toothbrush Handles, with and without Enzymes

Bombyx mori cocoons were shredded into small pieces using a paper shredding machine (bonsaii Cross Cut shredder 3S30 DIN P-4). The shredded pieces of cocoons were packed into predesigned molds. Glycerol was added to the cocoon pieces as a plasticizer (1%-5% wt) to promote the thermoplastic process. followed by hot pressing at 632 MPa and 145° C. for 15 minutes. After hot pressing, the samples were cooled to room temperature followed by machining to generate the desired shape as toothbrush handles. Enzyme-embedded toothbrush handles were prepared by mixing protease XIV powder (Millipore sigma) with silk cocoon pieces (1% wt) and thermoplastic molding followed by machining. The mixing of protease XIV powder with the silk cocoon pieces were achieved by sprinkling the protease powder on the silk cocoon pieces followed by packing the silk cocoon pieces in the mold for thermoplastic molding. The mixing of cellulose (microcrystalline, Millipore Sigma), HAP (<200 nm particle size, Millipore Sigma), chitosan (Millipore Sigma) to make composite plastics were achieved by mixing the material powders with silk cocoon pieces using a spatula and packing in the mold for thermoplastic molding.

### Biodegradation

Protease XIV was dissolved in DPBS solution as 1 mg/mL and 5 mg/mL. The thermoplastic molded silk cocoon silk bars (without embedded enzyme) were immersed into the solution and incubated at 37° C. The weight of the silk cocoon silk bars was weighed before and after incubation at various time point. Digital images were taken to record the morphological changes of the silk bars over time. The results are shown in FIGS. 3-4. The degradation of protease-embedded silk cocoon silk bars was conducted by immersing the silk bars into DPBS solution and incubated at 37° C. The weight was measured before and after incubation at various time points. For a third demonstration, the degradation in plant soil was conducted by immersing the silk bars without embedded enzyme into plants with 160 mL of room temperature water added each week. The degradation was monitored by taking digital photos every week for three weeks. The results are shown in FIGS. 5-6.

1. An article comprising a thermoplastically-molded silk cocoon from the species Bombyx mori, wherein the thermoplastically-molded silk cocoon has undergone plastic deformation.

2. The article of claim 1, the thermoplastically-molded silk cocoon further comprising a plasticizer.

3. The article of claim 2, wherein the plasticizer is present in the thermoplastically-molded silk cocoon in an amount by weight of between 1% and 50%.

4. The article of claim 2, wherein the plasticizer is selected from the group consisting of glycerol, water, low molecular weight silk fibroin, or sericin, or combinations thereof.

5. The article of claim 1, further comprising an additional biopolymer that is not present within the thermoplastically-molded silk cocoon.

6. The article of claim 1, further comprising at least one of a calcium salt, a cellulose, chitosan, chitin, hydroxyapatite (HAP) nanoparticles, a colorant, or an aromant.

7. The article of claim 1, further comprising an embedded protease selected from the group comprising protease XIV, a-chymotrypsin, proteinase K, and papain.

8. The article of claim 1, wherein a density of the article is between 0.6 g/cm³ and 1.4 g/cm³, including but not limited to at least 0.6 g/cm³, at least 1.0 g/cm³, at least 1.3 g/cm³, or at least 1.5 g/cm³, or at most 1.6 g/cm³, at most 1.0 g/cm³, or at most 0.6 g/cm³.

9. The article of claim 1, the article further comprising a density reducing additive having a lower bulk density than the thermoplastically-molded silk cocoon, thereby providing the article with a density that is lower than the bulk density of the thermoplastically-molded silk cocoon.

10. The article of claim 9, wherein the density reducing additive is at least one of entrapped gas, a hollow core, or a microcavity.

11. The article of claim 1, wherein a hardness of the article is between 30 and 100 on the Shore D scale.

12. The article of claim 1, wherein a flexural modulus of the article is between 1 and 40 GPa.

13. The article of claim 1, wherein a water absorbency of the article is between 1 wt. % and 30 wt. % according to the ASTM D570 test.

14. The article of claim 1, wherein the article has a brittle temperature between 0° C. and −80° C.

15. A method of making an article, comprising the following steps:
(a) shredding cocoons into silk strips;
(b) packing the silk strips into a mold;
(c) optionally adding between 1 wt. % and 50 wt. % of an additive to the silk strips in the mold; and
(d) pressing the silk strips in the mold at an elevated temperature, thereby forming the article.

16. The method of claim 15, wherein the additive is a plasticizer, cellulose, chitosan, chitin, hydroxyapatite, an enzyme, a colorant, an aromant, or a combination thereof, wherein the enzyme is selected from the group comprising protease XIV, a-chymotrypsin, proteinase K, and papain.

17-22. (canceled)

23. A method of making an article, the method comprising the following steps:
(a) shredding silk cocoons into silk strips;
(b) optionally coating the silk strips with between 1 wt. % and 50 wt. % of an additive;
(c) packing the silk strips into a mold; and
(d) pressing the silk strips in the mold at an elevated temperature, thereby forming the article.

24-31. (canceled)

32. The article of claim 1, wherein the article is dissolvable when contacted with a protease.

33. The article of claim 1, wherein the article is embedded in a moist plant soil.

34. (canceled)

35. The article of claim 7, wherein contacting the article with an aqueous solvent activates the embedded protease and dissolves the thermoplastically-molded silk cocoon.

36. (canceled)

37. (canceled)
